(19) United States
(12) Patent Application Publication
(10) Pub. No.: US 2021/0393368 A1
(43) Pub. Date: Dec. 23, 2021

(54) MEDICAL SHIELD TO PREVENT DISEASE TRANSMISSION AND RELATED METHODS

(71) Applicant: Alan Reyes, Fresno, CA (US)

(72) Inventor: Alan Reyes, Fresno, CA (US)

(21) Appl. No.: 17/344,933

(22) Filed: Jun. 10, 2021

Related U.S. Application Data
(60) Provisional application No. 63/039,992, filed on Jun. 17, 2020.

(51) Int. Cl. A61B 90/40

(52) U.S. Cl. CPC A61B 90/40

(57) ABSTRACT

The present invention provides a prophylactic drape shield system for use during a medical procedure concerning the upper body of a patient having a transmissible infection. For instance, the shield may be operable to prevent transmission of a viral infection such as novel corona virus from a patient to a medical worker who is performing an intubation or extubation procedure on the patient. The system may include a disposable drape shield made from a light, flexible, transparent and inexpensive material having an anterior panel and a posterior panel, the anterior panel including a semi-rigid face shield and at least one access port operable to allow the medical worker to pass his or her hand(s) inside the shield to perform the procedure. The system may therefore be easily stored and transported in large numbers for emergency situations such as a viral pandemic.

### FIELD OF THE INVENTION

The present invention relates generally to a protective shield system for preventing transmission of a contagious condition during a medical procedure, and more specifically to a prophylactic flexible shield for covering the upper body of a patient to prevent droplet-based transmission during intubation or extubation procedures, and methods for using the same.

### BACKGROUND OF THE INVENTION

The statements in this section merely provide background information related to the present disclosure and may not constitute prior art.

Infectious epidemics are an unfortunate, but regular occurrence. Whether the epidemic is due to a novel virus such as novel corona virus (2019-nCoV), or a previously controlled agent causing a local resurgence such as measles or tuberculosis, several people may end up at a medical facility in need of simultaneous medical care. A patient suffering from viral or bacterial infection may be highly contagious, and the infection may be transmitted via airborne droplets expelled from a patient while coughing, sneezing, or even simply breathing. Therefore, those people in the vicinity of the infected person are at high risk of contracting the infection. This is especially concerning in regard to the doctors, nurses, and technicians who may be performing a close-quarters medical procedure on the patient. Transmission of the infection to such medical professionals is exponentially damaging because it not only puts them in danger, but also increases the risk of infection to other potentially immune-compromised patients in a medical facility. Further, once the infection is recognized in the medical worker, he or she would need to be quarantined, reducing the capacity of the medical facility to treat an influx of patients during an emergency.

Patients with COVID-19 (or many other infectious diseases) often experience respiratory symptoms, and may require a medical procedure to treat those symptoms. These procedures typically require a medical worker to be in close vicinity to the patient's mouth and nose area. For example, in the case of respiratory failure, a medical worker may need to perform an intubation, which is characterized by manually opening the patient's mouth, adjusting the position of the patient's tongue with a laryngoscope, and inserting a tube into the patient's trachea. All the while the patient may be involuntarily choking or coughing, expelling droplets into the air around the medical worker's face and greatly increasing the risk of transmission despite the personal protective equipment (e.g., a mask and eye protection) worn by the medical worker.

Devices for providing additional protection against such transmission are needed to prevent transmission of droplets between a patient and a medical worker during a medical procedure.

### SUMMARY OF THE INVENTION

The present invention provides a prophylactic drape shield system for use during a medical procedure concerning the upper body of a patient having a transmissible infection. For instance, the shield may be operable to prevent transmission of a viral infection such as COVID-19 from a patient to a medical worker who is performing an intubation or extubation procedure on the patient. The system may include a disposable drape shield made from a light, flexible material having an anterior panel and a posterior panel, the anterior panel including a semi-rigid transparent face shield that allows the medical personnel to see the patient's mouth and airway clearly and at least one access port operable to allow the medical worker to pass his or her hand(s) inside the drape shield to perform an intubation or other procedure. The anterior panel may include an installation aid (e.g., a slit or cutout) to ease installation of the drape shield onto the patient.

The present invention may improve both the speed and outcome of a medical procedure while lowering costs for the medical facility. The drape shield of the present invention may be manufactured from inexpensive lightweight, foldable materials, wherein the drape shield may be folded into a substantially flat shape which is approximately the size of the face shield (e.g., approximately 12 inches long, approximately 10 inches wide, and approximately 1 inch in depth in the folded conformation) incorporated therein. Therefore, the drape shield may be available at a low cost and may be ordered in bulk by a medical facility. The foldable and lightweight nature of the drape shield may then allow it to be stored in bulk in multiple locations at a medical facility without taking up a substantial amount of space. The drape shield may be manufactured in an antiseptically and sealed in airtight packaging to provide a readily available sterile system wherever a patient requires treatment. The present invention may therefore be quickly accessed in an emergency situation. For instance, each surgery suite or examination room in a hospital may be stocked with several drape shields, all stored in a single cupboard or drawer. Also, a second drape shield may be accessed if the first one is splattered with body fluids or is otherwise contaminated, which may increase the risk of transmission or block the medical worker's view through the face shield. The low cost and ease of access of the present invention may even lead to a standard practice in which a first drape shield is used for an initial procedure (e.g., an intubation) and to increase sterility a second drape shield is used for a secondary or subsequent procedure (e.g., an extubation).

The benefits of the present invention are in contrast to conventional systems. For instance, a rigid shield system (e.g., a box) may represent a cost several times that of the present invention, may be cumbersome to store and transport, and would require sterilization after use, requiring significant costs in both labor and resources. Such devices are inefficient and disfavored. This is especially true when a facility desires to have multiple prophylactic systems available for the large number of patients which may need care during an epidemic-level emergency. Also, the drape shield of the present invention may be disposed of in a standard biohazard collection bin, which is generally already present in a medical procedure room.

The present invention may also be stored in bulk in a storage area separate from the procedure room, wherein several dozen drape shields may be held in a single box, each drape shield being easily portable in a hygienic fashion (e.g., in sealed packaging) by a single person to any area of need in a medical facility. Additionally, during a widespread health emergency, the present invention may be easily transported in large numbers from an established medical facility or storage location to a plurality of remote, temporary medical procedure locations which have been established in response to the emergency. After use, the present invention may be disposed of with the other medical waste.

The present system may also offer more flexibility, and thus greater precision, than conventional systems. The drape shield of the present invention may provide a flexible barrier wherein the ports for manual access by a medical worker are not rigidly stuck in a static position with respect to the patient's body. Rather, the medical worker may be able to move his or her arms, including the area which is in engaged with the access ports (e.g., the forearms), in a more natural way with a substantially full range of motion. The medical worker may therefore be able to interact with the patient's body and the instruments inside the drape shield in a more dexterous and precise manner, thereby maximizing the efficacy and safety of the procedure. For example, laryngoscopy prior to insertion of a breathing tube during an intubation procedure requires precise movement of the medical worker's hands and arms in order to accurately position the blade of the laryngoscope and make precise corrections. Without the full range of motion provided by the present invention, the blade may not be inserted or adjusted accurately, leading to patient injury and/or failure to properly place the breathing tube.

The present system may further provide greater sterility than conventional systems. Medical procedures involving the patient's mouth, nose (or other mucous membranes which may excrete infectious pathogens), may involve substantial expulsion forces that cause droplets to be produced and expelled in multiple directions from the patient's face. When installed over a patient the drape shield of the present invention provides improved protection against transmission of such droplets from the patient to the medical worker performing the procedure, and vice versa. For instance, inserting the blade of a laryngoscope behind the patient's tongue in order to visualize the larynx, or inserting the breathing tube through the larynx and into the trachea during an intubation, may trigger the patient's gag reflex and cause the patient to cough or expectorate saliva and/or mucus. Extubation, e.g., pulling the breathing tube back out, may induce a similar coughing reaction, or may simply toss droplets into the air from the force of pulling the tube out. Other procedures which may be aided by the present invention may include dental procedures (wherein saliva and dental tissue are expelled out of the mouth by a drill or polisher), rhinoplasty or restoration of the nasal cavities after a broken nose, and other similar procedures which may produce or expel droplets containing infectious pathogens.

As such droplets may be expelled from the patient in an expanding cloud, some droplets may be directed generally cranially. To counteract this, the access ports of the present invention may provide a snug fit around the medical worker's wrists, forearms, or upper arms. In some implementations each access port may include a cinching device (e.g., an elastic ring) operable to tighten around the forearm of the medical worker and prevent droplets expelled from the patient from escaping out the access port during a medical procedure. In some implementations, the cinching device may also include a sleeve of flexible material with an elastic ring at the distal end of the sleeve to engage with (e.g., tighten around) the arm of the medical worker. The sleeve may allow the medical worker to make adjustments of the position of his or her forearms toward or away from the patient without the elastic member sliding up and down the medical worker's arm. Thus, the cinching device of the present invention may increase sterility of the drape shield system without limiting the movement of the medical worker performing the procedure. Also, the flexible nature of the drape shield as a whole allows for lateral and vertical movement of the medical worker's arm while engaged with the access port, providing a full range of motion without compromising sterility.

Generally, the prophylactic drape shield system may include the following major components: a disposable drape shield made from a light, flexible material having an anterior panel and a posterior panel, the anterior panel including a semi-rigid face shield and at least one access port operable to allow the medical worker to pass his or her hand(s) inside the shield to perform the procedure. In some embodiments, the anterior panel may include an installation aid (e.g., a slit or cutout) to ease installation onto a prone patient. In some embodiments, the system may further include a cinching device at each access port to provide a tight fit with the arm of the medical worker and prevent droplets from escaping via the access port. In some embodiments, the system may further comprise an anti-bacterial and/or anti-viral coating on one or more surfaces of the drape shield.

The drape shield of the present invention may comprise an anterior panel (e.g., the upper layer of the shield which covers the front side of the patient) and a posterior panel (e.g., the lower layer of the shield which covers at least a portion of the back side of the patient). In some embodiments, the anterior and posterior panels may be either fused or adhered together at the proximal end of the shield and the two lateral sides of the shield.

The drape shield (e.g., the anterior panel and the posterior panel) may comprise any lightweight and flexible material which allows the drape shield to be foldable and easily transported and stored, but strong enough to resist puncture and tearing during installation over a patient and performance of a medical procedure. In some embodiments, the drape shield may comprise a transparent material, allowing the medical worker to see inside the drape shield and more accurately manipulate the patient and any needed medical instruments. In some embodiments, the drape shield may comprise a polyethylene material (e.g., low density polyethylene film). In some embodiments, the drape shield may comprise a thermoplastic polyurethane material such as transparent polyether TPU film. In other embodiments, the drape shield may comprise at least one of a plastic material, a nylon material, a nitrile material, a vulcanized rubber material, a latex material, and another material which is foldable and lightweight while retaining sufficient sheer strength and puncture resistance for prophylactic use during a medical procedure. The material of the drape shield also allows it to be folded, flexed, and manipulated as it is placed over and around a patient, allowing it to be deployed quickly and easily. In some embodiments, the anterior panel and/or posterior panel of the drape shield may comprise a thickness in a range from approximately 0.01 mm to approximately 3.0 mm, or any thickness or range of thicknesses therebetween (e.g., from approximately 0.02 mm to approximately 2.0 mm, from approximately 0.05 mm to approximately 1.5 mm, from approximately 0.1 mm to approximately 1.0 mm, from approximately 0.2 mm to approximately 0.5 mm, and the like). In some embodiments, the drape shield may comprise a thickness of approximately 0.3 mm.

In some embodiments, the anterior panel may comprise an extension (e.g., a flap) at the proximal end and each of the anterior sides. The flaps may have edges that can be joined together, such that a proximal edge of a lateral side flap and a lateral edge of the proximal end flap are adhered together or fused together in order to create vertical depth within the drape shield providing space to accept a patient's head and torso. With the patient inserted into three-dimensional space of the drape shield, the two lateral side flaps effectively act as vertical sidewalls and the proximal flap may act as a proximal sidewall between the anterior panel and the posterior panel. In some embodiments, each of the flaps may each extend approximately 6 inches out from the main body of the anterior panel. In some embodiments, each of the flaps may extend from the main body of the anterior panel by, e.g., about 10 cm to about 20 cm. The flexibility of the drape shield material and the three-dimensional space available for the patient within the drape shield allows the drape shield to be easily and quickly manipulated and positioned around the patient on table or gurney.

The perimeters flaps may be operable to fold over and be adhered to or fused with the posterior panel. In some embodiments, at least a portion of the perimeter of the anterior panel (with or without the flaps) may be adhered (e.g., in an air-tight manner) to at least a portion of the perimeter of the posterior panel. In such embodiments, the adherent may comprise a glue appropriate for the material of the anterior panel and posterior panel (e.g., a plastic glue for adhering polyethylene, or cyanoacrylate for adhering polyurethane). In other embodiments, the anterior panel may be fused in an airtight manner with the posterior panel (e.g., via heat fusion, friction fusion, pressure fusion, and the like). In yet other embodiments, the perimeter of the anterior panel may be connected to the perimeter of the posterior panel in another, similar manner.

The drape shield may comprise an overall length and width appropriate for accommodating the upper body of a person when installed thereon. In some embodiments, the width of the drape shield may be complementary to a width of a medical support surface (e.g., an operating table or gurney). In some embodiments, the drape shield may comprise a width in a range from approximately 2 feet to approximately 6 feet, or any width or range of widths therebetween (e.g., from approximately 2.5 feet to approximately 5.5 feet, from approximately 3 feet to approximately 5 feet, from approximately 3.5 feet to approximately 4.5 feet, and the like). In some embodiments, the drape shield may comprise a width of approximately 4 feet, including the two lateral side flaps of the anterior panel, or approximately 3 feet when not including the two lateral side flaps.

In some embodiments, the drape shield may comprise a length complementary to the length of the upper body of a patient. In some embodiments, the drape shield may comprise a length in a range from approximately 2 feet to approximately 6 feet, or any length or range of lengths therebetween (e.g., from approximately 2.5 feet to approximately 5.5 feet, from approximately 3 feet to approximately 5 feet, from approximately 3.5 feet to approximately 4.5 feet, and the like). In some embodiments, the drape shield may comprise a length of approximately 4.25 feet, including the proximal end flap of the anterior panel, or approximately 3.75 feet when not including the proximal end flap.

The drape shield (e.g., the anterior panel and the posterior panel) may comprise any shape operable to fit over the upper body of a patient lying on a medical support surface. The medical support surface may comprise at least one of an operating table, a gurney, an adjustable chair, and the like. In some embodiments, the drape shield may comprise a distal end (e.g., the end of the shield which provides an opening to fit over the patient) and a proximal end (e.g., the closed end which sits adjacent to the top of the patient's head when installed). The drape shield may comprise a substantially trapezoidal overall shape. In some embodiments, the trapezoidal shape may comprise a rectangular shape. In other embodiments, a distal end of the shield may comprise a greater length than the proximal end of the shield (e.g., providing the distal end with enough width to open up wide enough to easily fit over the patient during installation). In some embodiments, the distal side of the anterior panel may comprise an arcuate shape reaching from one lateral corner of the distal side to the other lateral corner. The arcuate shape may extend a length of the anterior panel at the longitudinal centerline of the drape shield in order to account for a change in three-dimensional shape of the drape shield while installed over the patient.

The material and shape of the drape shield may be operable to prevent droplets in the air from traversing a barrier (e.g., the anterior panel, the posterior panel, the face shield, or a cinching device) of the drape shield. The drape shield may thereby prevent transmission from a patient inside the drape shield to a medical worker outside the drape shield (and vice versa) of an infectious pathogen which is transmissible via droplets. The infectious pathogen may comprise at least one of a viral pathogen, a bacterial pathogen, a fungal pathogen and the like. The drape shield of the present invention may thus prevent transmission of conditions such as COVID-19, various other types of influenza, tuberculosis, and other similar conditions.

In some embodiments, the drape shield may comprise an antimicrobial coating on a surface of at least one of the anterior panel, the posterior panel, the face shield, and the cinching device(s). In some embodiments, the antimicrobial coating may cover an interior surface of the anterior panel. In some embodiments, the antimicrobial coating may cover an interior surface of face shield. In some embodiments, the antimicrobial coating may further cover an interior surface of the cinching device. In other embodiments, the antimicrobial coating may cover an exterior surface of the anterior panel. The antimicrobial coating may comprise any compound or material which is operable to reduce a viral, bacterial, and/or fungal load on a surface of the drape shield. In some embodiments, the antimicrobial coating may comprise a chemical operable to kill a bacterial or fungal cell or spore, or denature a virion (e.g., an alcohol, a bleach, an ammonium, and the like). In other embodiments, the antimicrobial coating may comprise a nanoscale biostatic layer operable to trap and prevent further movement of the pathogen. In yet other embodiments, the antimicrobial coating may comprise another similar compound or material operable to reduce a viral, bacterial, or fungal load on a surface.

The anterior panel may comprise a face shield. The face shield may comprise a transparent, semi-rigid material operable to hold its shape (e.g., substantially flat or having a gentle curve rather than sharp angles or wrinkles) more readily than the material of the remainder of the drape shield, providing a substantially clear and accurate view of the patient's face and head area. The face shield may thus be operable to allow for more precision in performing a medical procedure. The face shield may be arranged such that it is located substantially above the patient's head when the drape shield is installed over the patient. In some embodiments, the face shield may be located on a longitudinal centerline of the anterior panel. In some embodiments, the face shield may be located closer to the proximal end (e.g., near the head of the patient) of the drape shield than the distal end (e.g., the open end) of the drape shield. In some embodiments, the face shield may include a material comprising at least one of a polycarbonate, a propionate, an acetate, polyvinyl chloride, and polyethylene terephthalate glycol (PETG). In other embodiments, the face shield may comprise at least one of a polyethylene material and a thermoplastic polyurethane material.

The face shield may comprise any thickness, length, and width operable to provide sufficient strength—considering the material of the face shield—to hold its shape and provide a clear view of the area of the patient's body which is the subject of the medical procedure (e.g., for an intubation or extubation procedure, a portion of the lower half of the patient's face). The face shield may also be sufficiently flexible and resilient to allow some bending in handling (e.g., rough handling) in emergent situations while not sustaining plastic deformation, cracks, discolorations, or other damage that may obstruct the view through the shield or perforations therein. In some embodiments, the face shield may comprise a thickness in a range from approximately 0.01 mm to approximately 3.0 mm, or any thickness or range of thicknesses therebetween (e.g., from approximately 0.02 mm to approximately 2.5 mm, from approximately 0.05 mm to approximately 2.0 mm, from approximately 0.1 mm to approximately 1.5 mm, from approximately 0.5 mm to approximately 1.0 mm, and the like). In some embodiments, the face shield may comprise a thickness of approximately 1.5 mm.

In some embodiments, the face shield may comprise a length and width approximating a length and width of a patient's face. In some embodiments, the face shield may comprise a length (e.g., the dimension along the longitudinal centerline of the drape shield) in a range from approximately 5 inches to approximately 24 inches, and any length or range of lengths therebetween (e.g., from approximately 6 inches to approximately 20 inches, from approximately 8 inches to approximately 16 inches, from approximately 10 inches to approximately 14 inches, and the like). In some embodiments, the face shield may comprise a length of approximately 12 inches. In some embodiments, the face shield may comprise a width (e.g., the dimension running from one lateral side of the drape shield to the other) in a range from approximately 5 inches to approximately 24 inches, and any width or range of widths therebetween (e.g., from approximately 6 inches to approximately 18 inches, from approximately 8 inches to approximately 14 inches, from approximately 8 inches to approximately 12 inches, and the like). In some embodiments, the face shield may comprise a width of approximately 10 inches.

In some embodiments, the face shield may comprise a same material as a material of the anterior panel of the drape shield (e.g., a polyethylene or a polyurethane). In other embodiments, the face shield may comprise a different material than that of the anterior panel. In some embodiments, the face shield may be integral with the anterior panel. In other embodiments, the face shield may be manufactured separately from the anterior panel, and the anterior panel may comprise a face shield cutout having a shape complementary to a shape of the face shield. In such embodiments, the face shield may be adhered in an air-tight manner to a perimeter of the face shield cutout. In such embodiments, the adherent may comprise a glue appropriate for the material of the anterior panel and the face shield (e.g., a plastic glue for adhering polyethylene, or cyanoacrylate for adhering polyurethane). In other embodiments, the face shield may be fused in an airtight manner with the perimeter of the face shield cutout (e.g., via heat fusion, friction fusion, pressure fusion, and the like). In yet other embodiments, the face shield may comprise a snap-fit connection with the perimeter of the face shield cutout. In yet other embodiments, the face shield may be connected to the perimeter of the face shield cutout in another similar manner. In some embodiments, the perimeter of the face shield cutout may comprise a greater thickness than the surrounding material of the anterior panel in order to provide a stronger connection with the face shield.

The anterior panel may comprise at least one access port. The access port may be operable to allow a medical worker to manually access an interior of the drape shield while the drape shield is installed over the patient. In some embodiments, the at least one access port may comprise a plurality of access ports. In some embodiments, the at least one access port may comprise a pair of access ports arranged to allow the medical worker to access the patient's head and neck area with both of the medical worker's hands. In some embodiments, the pair of access ports may be arranged on opposing sides of a longitudinal centerline of the drape shield. In some embodiments, each of the pair of access ports may be located at an approximate midpoint between a lateral side of the face shield and a corresponding lateral side of the anterior panel (e.g., midway between the face shield and the edge of the drape shield). In some embodiments, the at least one access port may comprise a plurality of access ports on the same side of the longitudinal centerline of the drape shield.

The at least one access port may comprise a passage traversing the anterior panel of the drape shield, the passage comprising any perimeter shape operable to allow a medical worker manual access to an interior of the drape shield. In some embodiments, the at least one access port may comprise a substantially circular shape. In other embodiments, the at least one access port may comprise a substantially ovate shape. In yet other embodiments, the at least one access port may comprise another similar shape operable to allow the passage of the hand of a medical worker into an interior of the drape shield.

The at least one access port may be operable to provide little to no distance between a perimeter of the at least one access port and the hand or arm of the medical worker, thus limiting the passage of droplets into or out of the drape shield during a medical procedure. In some embodiments, the at least one access port may comprise a diameter in a range from approximately 2.5 inches to approximately 6 inches, and any diameter or range of diameters therebetween (e.g., from approximately 3 inches to approximately 5.5 inches, from approximately 3.5 inches to approximately 5 inches, from approximately 4 inches to approximately 4.5 inches, and the like).

In some embodiments, the at least one access port may comprise a cinching device allowing for a resilient rim around the access port such that arms of various sizes can pass through the access port and the cinching device closes around the arm to prevent any gaps. The cinching device may closely engage with an arm or hand of the medical worker during a medical procedure, thus preventing the escape of droplets through the access port. In some embodiments, the cinching device may comprise an elastic member, the elastic member being operable to increase in diameter in order to allow the passage of the hand of the medical worker, and automatically reduce in diameter to more tightly engage with at least one of the wrist, forearm, and upper arm of the medical worker during a medical procedure. In some embodiments, the elastic member may be permanently attached to a perimeter of the at least one access port (e.g., via at least one of an adhesive, a weld, threaded seam, and the like). In some embodiments, the elastic member may be nested within an enclosed, circular passage at a perimeter of the at least one access port. In other embodiments, the cinching member may comprise a substantially inelastic cord operable to be cinched around the arm of the medical worker.

In some embodiments, the cinching device may comprise an extension (e.g., a flexible sleeve or channel) for allowing movement of the medical worker's arm to be inserted through the access port and in proximity to the patient without the medical worker's body, clothing, or gear being exposed to potential contamination from the patient. In some embodiments, the extension may comprise a first end attached to a perimeter of the at least one access port and a closed second end. In other embodiments, the second end may be open, such that the extension is an open-ended sleeve structure, allowing the passage of a hand of the medical worker. In some embodiments, the extension may comprise a same material as the anterior panel, and the first end may be integral with the perimeter of the at least one access port (e.g., integral with the anterior panel). In other embodiments, the extension may comprise a permanent attachment to a perimeter of the anterior panel (e.g., an adhesive such as a glue appropriate for a material of the extension and the anterior panel, a weld, and another similar connection).

In some embodiments, the second end of the extension may comprise an elastic member (e.g., an elastic band), the elastic member being operable to increase in diameter in order to allow the passage of the hand of the medical worker, and automatically reduce in diameter to more tightly engage with at least one of the wrist, forearm, and upper arm of the medical worker during a medical procedure. In some embodiments, the elastic member may be permanently attached to a perimeter of the second end (e.g., via at least one of an adhesive, a weld, threaded seam, and the like). In some embodiments, the elastic member may be nested within an enclosed, circular passage at a perimeter of the second end.

The extension may comprise any lightweight and flexible material which allows the drape shield to be foldable and easily transported and stored, but strong enough to resist puncture and tearing during performance of a medical procedure. In some embodiments, the material of the extension may comprise a standard, opaque medical drape material (e.g., a standard blue paper or cloth material). In other embodiments the extension may comprise a transparent material. In some embodiments, the extension may comprise a polyethylene material (e.g., low density polyethylene film). In some embodiments, the extension may comprise a thermoplastic polyurethane material such as transparent polyether TPU film. In other embodiments, the extension may comprise at least one of a plastic material, a nylon material, a nitrile material, a vulcanized rubber material, a latex material, and another material which is foldable and lightweight while retaining sufficient sheer strength and puncture resistance for prophylactic use during a medical procedure. In some embodiments, the extension may comprise a thickness in a range from approximately 0.01 mm to approximately 3.0 mm, or any thickness or range of thicknesses therebetween (e.g., from approximately 0.02 mm to approximately 2.0 mm, from approximately 0.05 mm to approximately 1.5 mm, from approximately 0.1 mm to approximately 1.0 mm, from approximately 0.2 mm to approximately 0.5 mm, and the like). In some embodiments, the extension may comprise a thickness of approximately 0.3 mm. In some embodiments, the extension may comprise a length in a range from approximately 0.5 inches to approximately 24 inches (e.g., from approximately 1 inch to approximately 20 inches, from approximately 2 inches to approximately 18 inches, from approximately 3 inches to approximately 12 inches, from approximately 4 inches to approximately 10 inches, from approximately 5 inches to approximately 8 inches, and any length or range of lengths therebetween).

The posterior panel may comprise a gap allowing the posterior panel to be positioned around and/or under the patient. The gap may be arranged approximately along a longitudinal centerline of the posterior panel, allowing the posterior panel to be opened and subsequently wrapped around the sides of the patient. The gap's shape may be roughly complementary to a shape of the upper body of the patient. In some embodiments, the cutout may be substantially triangular in shape. In other embodiments, the cutout may be substantially rectangular in shape. In yet other embodiments, the cutout may comprise a shape approximating a silhouette of the patient. In some embodiments, the cutout may comprise a generally triangular shape having gently rounded corners, wherein two sides of the perimeter of the triangular shape may be wrapped around and tucked under the shoulders of the patient in order to secure the drape shield in place. In some embodiments, the posterior panel may be comprised of a flexible, foldable, semi-rigid material that provides structure to the drape shield. In such embodiments, the semi-rigid material may aid in the quick deployment of the drape shield around the head and torso of the patient. For example, the medical worker may grasp the two lateral portions of the gap in the posterior panel to evert the lateral portions outward to widen the gap to around the patient, and then once the lateral portions are positioned at the patient's back, allowing the resilient, semi-rigid material to resume its shape, and, e.g., tuck under the patient's shoulders. In such examples, the semi-rigid material may allow the medical worker to more quickly position the drape shield securely over the patient, without having to spend significant time tucking edges under the patient to anchor the drape shield.

Several embodiments are discussed below, but the example embodiments shall not be interpreted as an exhaustive list. One with ordinary skill in the art will recognize that the scope of the present invention includes further variations and equivalents to the specific examples described herein.

In some implementations, the present invention may provide a prophylactic drape shield for use during a medical procedure, the system comprising: a drape shield having an anterior panel and a posterior panel; at least one access port; and a face shield. In some implementations, the drape shield is operable to slide over the head and upper body of a patient while the patient is located on a medical support structure. In some implementations, the drape shield is operable to provide a prophylactic barrier against transmission of an infectious disease from the patient to a medical worker performing a medical procedure on the patient. In some implementations the drape shield is operable to prevent droplets expelled from the mouth or nose the patient during an intubation or extubation procedure from contacting the head of the medical worker. In some implementations, face shield and the at least one access port are arranged on the anterior panel. In some implementations, the face shield is arranged on a centerline of the anterior panel and adjacent to a head of a patient when the drape shield is installed over the patient, and the at least one access port comprises a first access port and a second access port, the first and second access ports being arranged on opposite sides of the centerline. In some implementations, the first and second access ports are operable to allow manual access to an interior of the drape shield on opposing sides of the head of the patient. In some implementations, the face shield comprises a semi-rigid, transparent material operable to provide a clear view of the head of the patient. In some implementations, the at least one access port is operable to allow a hand of a medical worker to traverse the anterior panel. In some implementations, the at least one access port comprises a cinching device. In some implementations, the cinching device comprises a sleeve. In some implementations, the cinching device comprises an elastic member. In some implementations, the drape shield comprises a substantially transparent material. In some implementations, posterior panel comprises at least one of a slit and a cutout for allowing the drape shield to be easily installed over an upper body of a patient. In some implementations, the drape shield comprises a lightweight, substantially flexible material, and the face shield comprises a lightweight, semi-rigid material. In some implementations, the drape system is easily transported and stored in large numbers, and quickly and efficiently deployed in an emergency.

In some implementations, the present invention may provide a drape shield system for prophylactic use during an intubation or extubation procedure, the drape shield comprising an anterior panel and a posterior panel, the anterior panel having a transparent, semi-rigid face shield for providing a clear view of a patient's head, and a plurality of access ports arranged to allow manual access on either side of the patient's head, the plurality of access ports each comprising a cinching device, and the posterior panel comprising a cutout for easily installing the drape shield over the patient. In some implementations, the anterior panel and the posterior panel each comprise a lightweight, flexible material and the system is easily transported and stored in large numbers, and quickly and efficiently deployed in an emergency.

A method of using the drape shield of the present invention may comprise the following steps: providing a drape shield for prophylactic use during a medical procedure, the drape shield comprising an anterior panel and a posterior panel, the anterior panel having a transparent, semi-rigid face shield for providing a clear view of a patient's head, and a plurality of access ports arranged to allow manual access on either side of the patient's head, the plurality of access ports each comprising a cinching device, and the posterior panel comprising a cutout for easily installing the drape shield over the patient; installing the drape shield over an upper body of a patient; and manually accessing an interior of the drape shield via the plurality of access ports. In some embodiments, the method may further comprise the step of performing a medical procedure on the patient. In some embodiments, the method may further comprise the step of disposing of the drape shield.

It is an object of the present invention to provide a prophylactic system operable to reduce the chance of transmission of infection between a patient and a medical worker during a medical procedure.

It is a further object of the present invention to provide a medical drape shield system which is lightweight, foldable, and portable in large numbers, and thus easy to store and transport to places of need.

It is a further object of the present invention to provide a medical drape shield system which that is flexible and easily and quickly manipulated and positioned around the patient on table or gurney.

It is a further object of the present invention to provide a medical drape shield system which is inexpensive and disposable.

It is a further object of the present invention to provide a medical drape shield system which allows a medical worker to have a full range of motion during a medical procedure.

It is a further object of the present invention to provide a medical drape shield system with a manual access port which is operable to fit over various sized hands and arms and provide a tight fit thereon, thus preventing droplets from escaping through the access port.

If is a further object of the present invention to provide a medical drape shield system which provides a clear and unobstructed view of the patient's head and neck area.

The above-described objects, advantages and features of the invention, together with the organization and manner of operation thereof, will become apparent from the following detailed description when taken in conjunction with the accompanying drawings, wherein like elements have like numerals throughout the several drawings described herein. Further benefits and other advantages of the present invention will become readily apparent from the detailed description of the preferred embodiments.

### BRIEF DESCRIPTION OF THE DRAWINGS

FIG. 1A provides a perspective view of a drape shield system, according to an embodiment of the present invention.

FIG. 1B provides top-down and side views, respectively, of a drape shield system, according to an embodiment of the present invention.

FIG. 1C provides top-down and side views, respectively, of a drape shield system, according to an embodiment of the present invention.

FIG. 2A provides top-down perspective and bottom-up perspective views, respectively, of a drape shield system, according to an embodiment of the present invention.

FIG. 2B provides top-down perspective and bottom-up perspective views, respectively, of a drape shield system, according to an embodiment of the present invention.

FIG. 2C provides a top-down view of a drape shield system, according to an embodiment of the present invention.

FIG. 2D provides a side view of a drape shield system, according to an embodiment of the present invention.

FIG. 2E provides a bottom-up view of a drape shield system, according to an embodiment of the present invention.

FIG. 2F provides a rear view (e.g., from the foot of the patient) of a drape shield system, according to an embodiment of the present invention.

FIG. 2G provides a side view of a drape shield system, according to an embodiment of the present invention.

FIG. 2H provides a front view (e.g., from the head of the patient) of a drape shield system, according to an embodiment of the present invention.

FIG. 3A provides a top-down view of an anterior panel and posterior panel of a drape shield system, according to an embodiment of the present invention.

FIGS. 3B and 3C provide a top-down view and a side view of a drape shield system in a folded conformation, according to an embodiment of the present invention.

FIG. 3D provides a side view of a plurality of drape shield systems stored in a folded conformation, according to an embodiment of the present invention.

FIG. 4A provides perspective views of an anterior panel of a drape shield system, according to an embodiment of the present invention.

FIG. 4B provides perspective views of an anterior panel of a drape shield system, according to an embodiment of the present invention.

### DETAILED DESCRIPTION

Reference will now be made in detail to certain embodiments of the invention, examples of which are illustrated in the accompanying drawings. While the invention will be described in reference to these embodiments, it will be understood that they are not intended to limit the invention. To the contrary, the invention is intended to cover alternatives, modifications, and equivalents that are included within the spirit and scope of the invention. In the following disclosure, specific details are given to provide a thorough understanding of the invention. However, it will be apparent to one skilled in the art that the present invention may be practiced without all of the specific details provided.

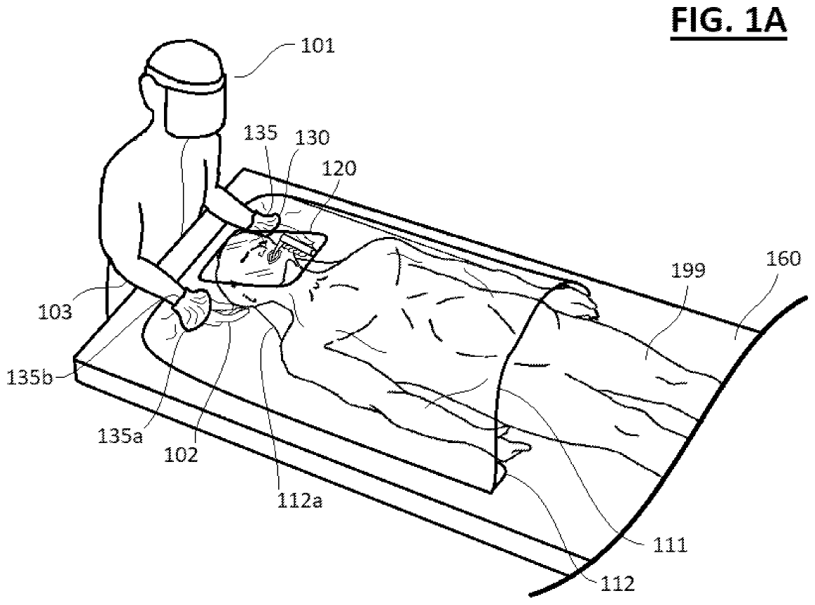

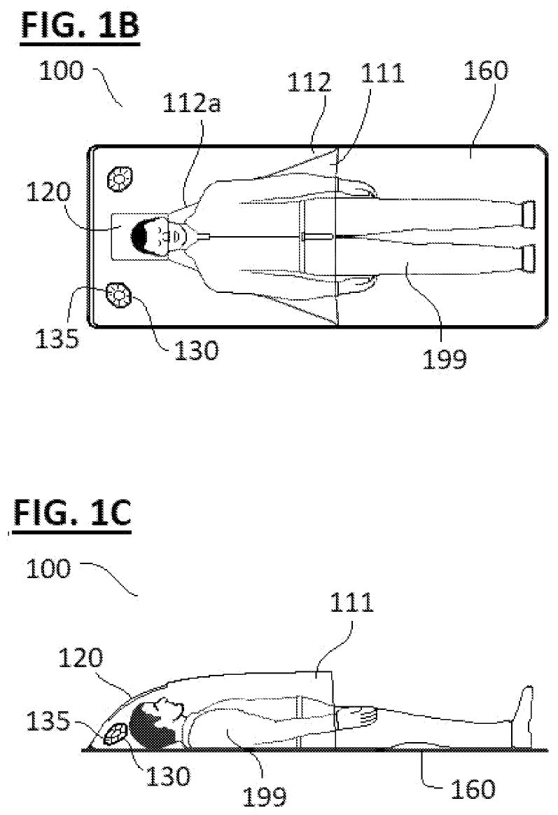

The present invention concerns a prophylactic drape shield system 100 for use during a medical procedure concerning the upper body of a patient having a transmissible infection. As seen in FIGS. 1-4B, the system may include the following major components: a disposable drape shield 110 made from a light, flexible material having an anterior panel 111 and a posterior panel 112, the anterior panel 111 including a semi-rigid face shield 120 and at least one access port 130 operable to allow the medical worker 101 to pass his or her hand(s) 102 inside the shield 110 to perform the procedure, and the anterior panel 112 including an installation aid 140 (e.g., a slit or cutout) to ease installation onto a prone patient 199. In some embodiments, the system may further include a cinching device 135 at each access port 130 to provide a tight fit with the arm 103 of the medical worker 101 and prevent droplets from escaping via the access port 130.

The drape shield 110 of the present invention may comprise an anterior panel 111 (e.g., the upper layer of the shield 110 which covers the front side of the patient 199) and a posterior panel 112 (e.g., the lower layer of the shield 110 which covers at least a portion of the back side of the patient 199). The drape shield 110 (e.g., the anterior panel 111 and the posterior panel 112) may comprise any lightweight and flexible material which allows the drape shield 110 to be foldable and easily transported and stored, but strong enough to resist puncture and tearing during installation over a patient 199 and performance of a medical procedure. The medical support surface may comprise an operating table 160. The drape shield 110 may comprise a transparent material (see FIGS. 1A-1C), allowing the medical worker 101 to see inside the drape shield 110 and more accurately manipulate the patient 199 and any needed medical instruments 198. In some embodiments, the drape shield 110 may comprise a distal end 110b (e.g., the end of the shield which provides an opening to fit over the patient 199) and a proximal end 110a (e.g., the closed end which sits adjacent to the top of the patient's 199 head when installed). The drape shield 110 may comprise a rectangular overall shape.

The posterior panel 112 may comprise an installation aid 112a, the installation aid may comprise a cutout operable to ease installation of the drape shield 110 over the upper body of a patient 199 lying on a medical surface 160 (e.g., an operating table). The cutout may comprise a shape complementary to a shape of the upper body of the patient 199, e.g., a general triangular shape having gently rounded corners, wherein two sides of the triangular shape may be wrapped around and tucked under the shoulders of the patient 199 in order to secure the drape shield in place over the patient (see FIGS. 1A-1C).

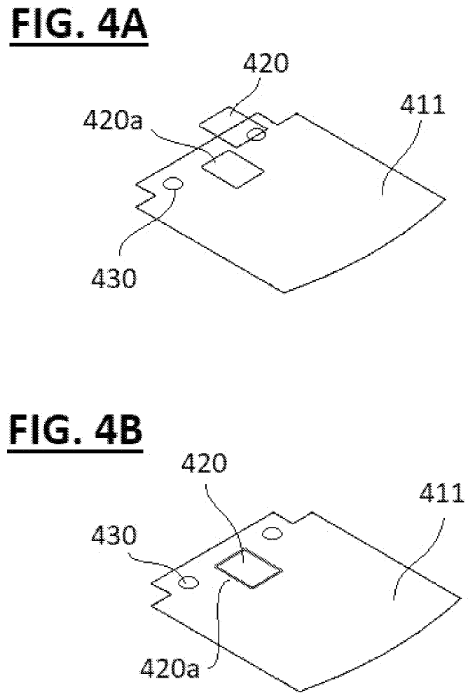

The anterior panel 111 may comprise a face shield 120, the face shield 120 comprising a transparent, semi-rigid material operable to hold its shape more readily than the material of the remainder of the drape shield 110, thereby providing a substantially clear view of the patient's 199 face and head area in order to more precisely perform a medical procedure. The face shield 120 may be arranged such that it is located substantially above the patient's 199 head when the drape shield 110 is installed. The face shield 120 may comprise a length of approximately 12 inches and a width of approximately 10 inches. As seen in FIG. 4A, the face shield 420 may be manufactured separately from the anterior panel 411, and the anterior panel 411 may comprise a face shield cutout 420a having a shape complementary to a shape of the face shield 420. The face shield 420 may be adhered in an air-tight manner to a perimeter of the face shield cutout 420a (see FIG. 4B).

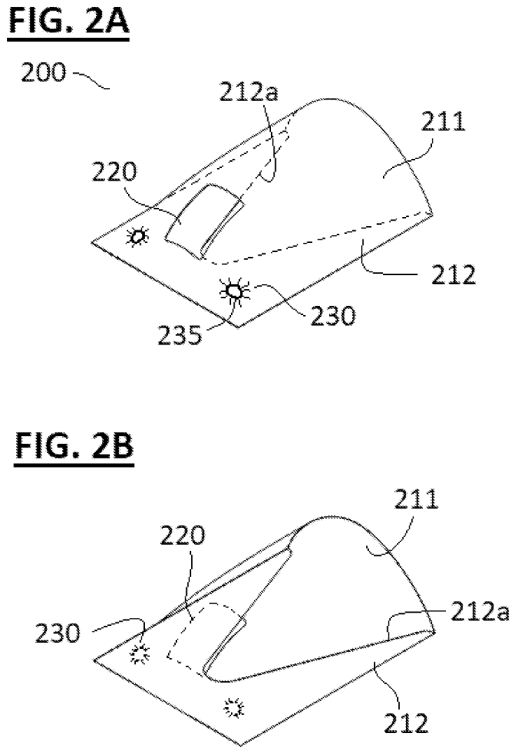

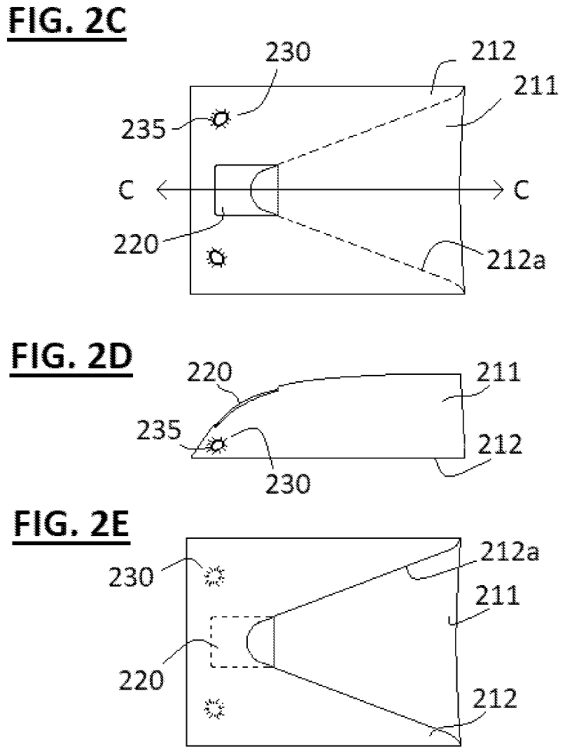

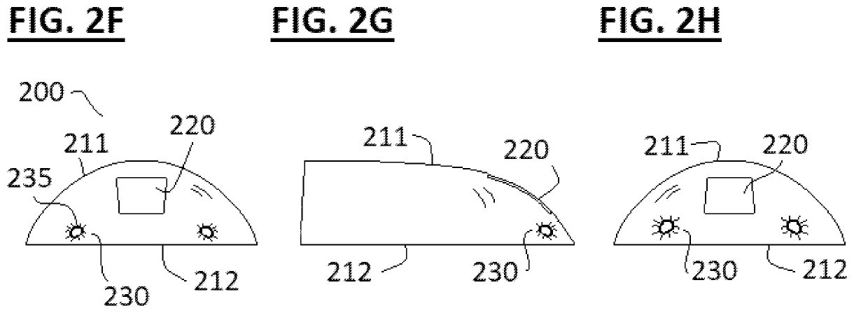

The anterior panel 111 may comprise a pair of access ports 130 operable to allow a medical worker 101 to manually access an interior of the drape shield 110 while the drape shield 110 is installed over the patient 199. The pair of access ports may be arranged to allow the medical worker to access the patient's head and neck area with both of the medical worker's hands 102. The pair of access ports may be arranged on opposing sides of a longitudinal centerline C (see FIG. 2C) of the drape shield 110.

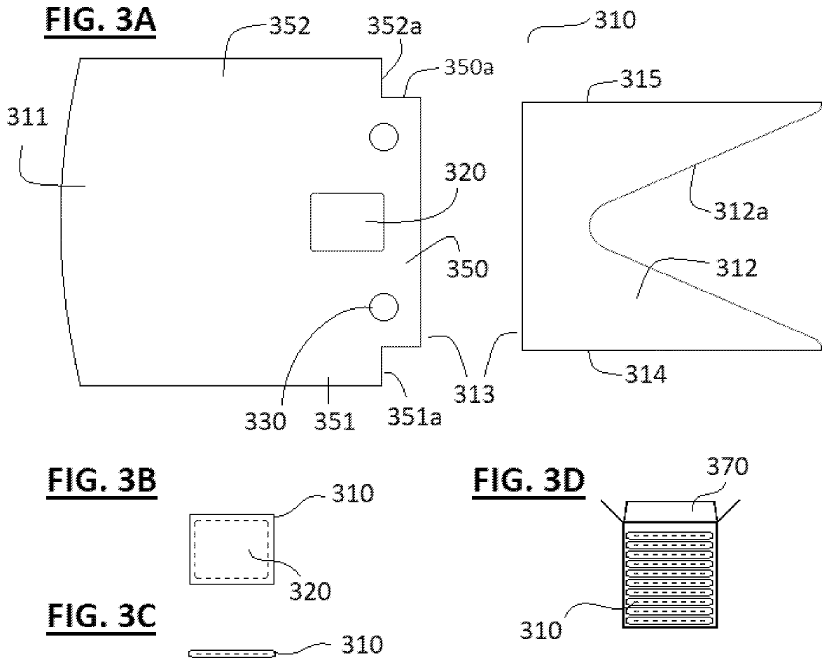

Each access port 130 may comprise a passage in the anterior panel 111 operable to allow a medical worker manual access to an interior of the drape shield. As seen in FIGS. 3A-3D and 4A-4B, in some embodiments each the access port 330 may comprise a simple passage having substantially circular shape and a diameter of approximately 4.13 inches.

In other embodiments, (see, e.g., FIGS. 1A-1C) each access port 130 may comprise a cinching device 135 for cinching a diameter of the access port to more tightly engage with an arm 103 or hand 102 of the medical worker 101 during a medical procedure. As best seen in FIG. 1A, the cinching device 135 may comprise an extension (e.g., a flexible sleeve or channel) for allowing movement of the medical worker's arm 103 either further into or partially out of the access port 130, thus allowing the medical worker 101 a greater range of motion during the medical procedure without disturbing (e.g., pulling or tugging on) the anterior panel 111. The extension may comprise a first end 135a attached to a perimeter of the at least one access port 130 and a second end 135b comprising an opening for allowing the passage of a hand 102 of the medical worker 101. The second end 135b of the extension may comprise an elastic member (e.g., a ring or band), the elastic member being operable to increase in diameter in order to allow the passage of the hand 102 of the medical worker 101, and automatically reduce in diameter to more tightly engage with the forearm 102 of the medical worker 101 during a medical procedure. The extension 135 may comprise any lightweight and flexible material which allows the drape shield 110 to be foldable and easily transported and stored, but strong enough to resist puncture and tearing during performance of a medical procedure.

As seen in FIGS. 2A-2F, in some embodiments the cinching device 335 may not comprise an extension, but rather may comprise an elastic member attached to a perimeter of the at least one access port 330. The elastic member 135 may be nested within an enclosed, circular passage at a perimeter of the at least one access port 130.

As seen in FIG. 3A, the drape shield 310 may comprise anterior panel 311 and posterior panel 312 which may be formed separately and subsequently fused together at the proximal end 313 of the shield 310 and the two lateral sides 314, 315. The anterior panel 311 may comprise a proximal flap 350, a first lateral flap 351, and a second lateral flap 352. The flaps 351, 352, 353 may be brought together at their edges, such that (i) a proximal edge 351a of a lateral side flap 351 and a lateral edge 350a of the proximal end flap 350 may be connected (e.g., adhered or fused together), and (ii) a proximal edge 352a of a lateral side flap 352 and a lateral edge 350b of the proximal end flap 351 may be connected (e.g., adhered or fused together) in order to create a seam therebetween and to create a vertical depth to the drape shield into which a patient can be situated. The anterior panel 311 may thereby have a three-dimensional space (e.g., a height, in addition to a length and a width) into which the patient may be inserted. The drape shield 310 may thereby more easily accommodate a patient's 199 upper body between the anterior panel 311 and the posterior panel 312. The flexible material of the anterior panel 311 allows the anterior panel 311 and the drape shield generally to be easily and quickly manipulated and positioned around the patient on table or gurney.

As seen in FIGS. 3B and 3C, the drape shield 310 may be manufactured from inexpensive lightweight, foldable material, wherein the drape shield 310 may be folded into a substantially flat shape which is only slightly larger than the size of the face shield 320 (e.g., approximately 12 inches long, approximately 10 inches wide, and approximately 1 inch or less in depth in the folded conformation). Therefore, the drape shield 310 may be operable to be stored in bulk in a storage space 370 (e.g., a box stored in a cupboard of a surgical suite) at a medical facility without taking up a substantial amount of space (see FIG. 3D). The drape shield 310 may therefore be quickly accessed in an emergency situation, and a replacement drape shield may be accessed if the first drape shield becomes contaminated or otherwise unusable.

The foregoing descriptions of specific embodiments of the present invention have been presented for purposes of illustration and description. They are not intended to be exhaustive or to limit the invention to the precise forms disclosed, and many modifications and variations are possible in light of the above teaching. The embodiments were chosen and described in order to best explain the principles of the invention and its practical application, to thereby enable others skilled in the art to best utilize the invention and various embodiments with various modifications as are suited to the particular use contemplated.

What is claimed is:

1. A prophylactic drape system for use during a medical procedure, the system comprising:
a. a drape shield having an anterior panel and a posterior panel;
b. at least one access port; and
c. a face shield.

2. The system of claim 1, wherein said drape shield is operable to slide over the head and upper body of a patient while said patient is located on a medical support structure.

3. The system of claim 1, wherein said drape shield is operable to provide a prophylactic barrier against transmission of an infectious disease from said patient to a medical worker performing a medical procedure on said patient.

4. The system of claim 3, wherein said drape shield is operable to prevent droplets expelled from the mouth or nose said patient during an intubation or extubation procedure from contacting the head of said medical worker.

5. The system of claim 1, wherein said face shield and said at least one access port are arranged on said anterior panel.

6. The system of claim 5, wherein said face shield is arranged on a centerline of said anterior panel and adjacent to a head of a patient when said drape shield is installed over said patient, and said at least one access port comprises a first access port and a second access port, said first and second access ports being arranged on opposite sides of said centerline. The system of claim 6, wherein said first and second access ports are operable to allow manual access to an interior of said drape shield on opposing sides of said head of said patient.

8. The system of claim 6, wherein said face shield comprises a semi-rigid, transparent material operable to provide a clear view of said head of said patient.

9. The system of claim 1, wherein said at least one access port is operable to allow a hand of a medical worker to traverse said anterior panel.

10. The system of claim 9, wherein said at least one access port comprises a cinching device.

11. The system of claim 10, wherein said cinching device comprises a sleeve.

12. The system of claim 10, wherein said cinching device comprises an elastic member.

13. The system of claim 10, wherein said drape shield comprises a substantially transparent material.

14. The system of claim 1, wherein said posterior panel comprises at least one of a slit and a cutout for allowing said drape shield to be easily installed over an upper body of a patient.

15. The system of claim 1, wherein said drape shield comprises a lightweight, substantially flexible material, and said face shield comprises a lightweight, semi-rigid material.

16. The system of claim 14, wherein said drape system is easily transported and stored in large numbers, and quickly and efficiently deployed in an emergency.

17. A drape shield system for prophylactic use during an intubation or extubation procedure, said drape shield comprising an anterior panel and a posterior panel, said anterior panel having a transparent, semi-rigid face shield for providing a clear view of a patient's head, and a plurality of access ports arranged to allow manual access on either side of said patient's head, said plurality of access ports each comprising a cinching device, and said posterior panel comprising a cutout for easily installing said drape shield over said patient.

18. The drape shield of claim 17, wherein said anterior panel and said posterior panel each comprise a lightweight, flexible material and said system is easily transported and stored in large numbers, and quickly and efficiently deployed in an emergency.

19. A method of using a medical drape shield system, comprising the steps of:
a. providing a drape shield for prophylactic use during a medical procedure, said drape shield comprising an anterior panel and a posterior panel, said anterior panel having a transparent, semi-rigid face shield for providing a clear view of a patient's head, and a plurality of access ports arranged to allow manual access on either side of said patient's head, said plurality of access ports each comprising a cinching device, and said posterior panel comprising a cutout for easily installing said drape shield over said patient;
b. installing said drape shield over an upper body of a patient; and
c. manually accessing an interior of said drape shield via said plurality of access ports.

20. The method of claim 19, further comprising the step of performing at least one of an extubation procedure and an intubation procedure.
